(19) United States
(12) Patent Application Publication
(10) Pub. No.: US 2011/0045557 A1
(43) Pub. Date: Feb. 24, 2011

(54) Novel Fusion Carbonic Anhydrase/Cellulose Binding Polypeptide Encoded by a Novel Hybrid Gene, and Method of Creating and Using the Same

(76) Inventors: Mohammed Ataai, Pittsburgh, PA (US); Robert Dilmore, Irwin, PA (US); Yee Soong, Monroeville, PA (US); Richard Koepsel, Pittsburgh, PA (US); Zhu Liu, Pittsburgh, PA (US)

Correspondence Address: UNITED STATES DEPARTMENT OF ENERGY, 1000 INDEPENDENCE AVENUE, S.W., ATTN: GC-62 (CHI), MS 6F-067, WASHINGTON, DC, 20585-0162, US

(21) Appl. No.: 11/829,359

(22) Filed: Jul. 27, 2007

(51) Int. Cl. C12P 7/62; C12N 9/02; C07H 21/04; C12N 15/63; C12N 11/12

(52) U.S. Cl. 435/135; 435/189; 536/23.4; 435/320.1; 435/179

(57) ABSTRACT

The invention relates a novel hybrid carbonic anhydrase catalyst with the potential to contribute significantly to meeting targeted reductions in greenhouse gas emissions. In a preferred embodiment of the present invention at least a portion of a cellulose binding domain (CBD) of a protein is fused to another protein, carbonic anhydrase, (CA) to create a new multi-functional protein which can bind tightly to cellulose while maintaining its native catalytic ability to process CO₂. The resulting CA-CBD hybrid polypeptide can be immobilized to a cellulose support and used to cost-effectively capture CO₂ from gas streams and other CO₂-rich environs.

### U.S. GOVERNMENT RIGHTS

The United States Government has rights in this invention pursuant to the employer-employee relationship between the U.S. Government some of the inventors.

### TECHNICAL FIELD

The invention relates to a novel fusion carbonic anhydrase (CA)/cellulose binding domain (CBD) protein produced from a novel gene and method of creating and using the same. The resulting CA-CBD hybrid polypeptide can be immobilized to a cellulose support and used to cost-effectively process CO₂ from gas streams and other CO₂-rich environs.

### SEQUENCE LISTINGS

The sequence listing information contained in the text file (txt) named: s108423_ST25.txt, created on Mar. 22, 2007, having a size of 41 KB, filed concurrently with the instant application is hereby incorporated by reference in its entirety.

### BACKGROUND OF THE INVENTION

An ever-increasing body of scientific evidence suggests that anthropogenic release of CO₂ is leading to a rise in global atmospheric temperatures that may, if unabated, could result in catastrophic climate and ecosystem change. One of the largest sources of CO₂ is coal-fired power plants. As compared with CO₂ from mobile sources, capture and sequestration of CO₂ from coal-fired power plants and other large point sources, is technically feasible. Sequestration of CO₂ from such sources would contribute significantly to reduction in global CO₂ emissions.

The concentration of CO₂ in coal-fired power plant flue gas varies from 10-15% as a function of process operating conditions. Most proposed CO₂ sequestration schemes require concentrated liquid or supercritical CO₂ streams. Available technologies for capture and separation of CO₂ from the flue gas and other sources include the use of solid sorbents for pressure swing adsorption (PSA), temperature swing adsorption (TSA) and concentration swing adsorption (CSA), the employment of liquid amines for absorption, and membrane separation process. These approaches have been used for removal of CO₂ from closed environments and the natural gas liquefaction process.

Aqueous amine CSA capture is considered to be the state of the art technology for CO₂ capture for pulverized coal (PC) power plants. Analysis conducted at the National Energy Technology Laboratory (NETL) shows that amine capture of CO₂ and compression raises the cost of electricity from a newly-built supercritical PC power plant by 84 percent, from 4.9 cents/kWh to 9.0 cents/kWh. NETL's goal for advanced CO₂ capture systems with subsequent compression is an electricity cost increase of no more than about 20 percent as compared to a no-capture case for a newly constructed power plant. In addition to the cost, amine scrubbing solutions are highly corrosive, presenting potential operation and maintenance difficulty and spent scrubber solution waste management issues.

Recently the present researchers began investigating the use of biological enzymes to remove and/or treat carbon dioxide and/or other contaminants from a gas stream. Enzymes can be utilized as free catalysts in solution or in an immobilized form attached to an insoluble support matrix. An advantage of immobilized enzyme reactors compared to enzymes in free solution is that the immobilization keeps the enzyme in the reactor and thus it is not lost in the product stream. Also, in many cases enzymes that are immobilized within a porous matrix are protected from harmful environmental stresses in the reactor and are more stable, retaining their activity for much longer periods of operation that those lose in solution. There are various support materials (generally employed in beaded form) and different modes of attachment for enzyme immobilization. Selection of a support material for immobilization of a particular enzyme is driven by several factors, including: support material cost, ease of enzyme immobilization, potential to regenerate support material, and the effect of immobilization on specific activity of the enzyme. Enzyme immobilization can be achieved either by physical methods, such as enzyme entrapment within an insoluble gel matrix of a porous membrane, or through chemical binding of the enzyme with functional groups of the activated support materials. Currently enzyme immobilization is expensive and not cost-effective for use on a large scale processes.

In order to meet the growing demand for a cost-effective means for removing CO₂ from a gas stream or other CO₂ rich environ, a new system and method is needed.

### SUMMARY OF THE INVENTION

One aspect of the present invention represents a new CO₂ capture system based on a novel immobilized carbonic anhydrase catalyst with the potential to contribute significantly to meeting targeted reductions in greenhouse gas emissions.

In a preferred embodiment of the present invention at least a portion of a cellulose binding domain (CBD) of a protein is fused to another protein, carbonic anhydrase, (CA) to create a new bifunctional protein which can bind tightly to cellulose while maintaining its native catalytic ability to process CO₂.

The invented fusion protein, referred to as CA-CBD, utilizes efficient binding of CBD to cellulose for cost-efficient immobilization of the CA on to cellulose-based supports. Cellulose is a superb matrix for enzyme immobilization due to its low cost and exceptional physical properties.

The use of a CA-CBD fusion versus the native CA may eliminate the need for enzyme purification and enables the use of a very inexpensive matrix (cellulose) for enzyme immobilization. The use of CA-CBD also eliminates the need to use immobilization chemistry to fix the enzyme hence it will dramatically reduce the overall process cost.

One or more of the invented CA-CBD polypeptides can be used as part of a new CO₂ capture system and process based on a CA-CBD hybrid immobilized on a cellulose-based matrix. The CA catalyzes at a very high rate (˜1.3×10⁶CO₂ molecules per second) the hydration and dehydration of dissolved CO₂ as shown in FIG. 5.

Following absorption of CO₂ from flue gas, HCO₃⁻ can be managed in several ways. CO₂ can be contacted with calcium and/or magnesium-bearing solutions to promote mineralization, it can be re-evolved in a CO₂ concentrated sweep stream through concentration gradient and compressed to a liquid and shipped to a long-term sequestration destination. Alternatively, it can be converted, via other biological processes, to mineral carbonates or fixed as simple organics.

One embodiment of the present invention generally relates to a fusion polypeptide encoding for a carbonic anhydrase with the ability to bind to cellulose.

One embodiment of the present invention generally relates to a fusion polypeptide comprising: an amino acid sequence that is at least 85%, or at least 90%, or at least 91%, or at least 92%, or at least 93%, or at least 94%, or at least 95%, or at least 98% identical to that of (SEQ ID NO: 8).

One embodiment of the present invention generally relates to a fusion polypeptide comprising: a first polypeptide sequence and second polypeptide sequence, wherein the fusion protein has an amino acid sequence of (SEQ ID NO: 8), or a functional variant, biologically active fragment or derivatives thereof.

Another embodiment of the invention relates a fusion polypeptide, wherein the polypeptide is a functional equivalent or biologically active fragment comprising at least 500 continuous amino acids of (SEQ ID NO: 8).

Yet another embodiment of the invention relates a fusion polypeptide, wherein the polypeptide has an amino acid sequence of (SEQ ID NO: 8) with conservative amino acid substitutions.

Yet another embodiment of the invention relates a fusion polypeptide, wherein the polypeptide has an amino acid sequence of (SEQ ID NO: 8) with 1-500 conservative amino acid substitutions, preferably between 1-100 conservative amino acid substitutions, more preferably between about 1-20 conservative amino acid substitutions.

One embodiment of the invention is an isolated and purified nucleic acid comprising the nucleotide sequence in (SEQ ID. NO: 1).

Another embodiment of the invention relates to an isolated nucleic acid comprising the nucleotide sequence in (SEQ ID. NO: 1) which encodes a CA-CBD fusion polypeptide, and functional equivalents, biologicially active derivatives and/or fragments thereof.

One embodiment of the present invention generally relates to a fusion poly nucleotide that is at least 95% identical to that of SEQ ID NO: 1.

Another embodiment of the invention is a nucleic acid sequence that is capable of hybridizing under stringent conditions to a nucleotide sequence found in (SEQ ID NO: 1) or its complements.

Another aspect of the invention is an RNA molecule that includes the nucleotide sequence set forth in (SEQ ID NO: 7), or functional and degenerate variants thereof, wherein Uracil (U) is substituted for Thymine (T).

Also included in the invention are nucleotides carrying modifications such as substitutions, small deletions, insertions or inversions which still encode proteins having substantially the same activity as the protein of (SEQ ID NO: 8). Included are nucleic acid molecules having a sequence is at least 85%, or at least 90%, or at least 91%, or at least 92%, or at least 93%, or at least 94%, or at least 95%, or at least 98% identical to that of SEQ ID NO: 1.

Yet another embodiment of the invention relates to a method of using the hybrid DNA sequences to express the polypeptides which they encode.

Yet another embodiment of the invention relates to an expression vector comprising the gene encoded by (SEQ ID NO. 1) operably inked to an expression control sequence.

Yet another embodiment of the invention relates to an expression vector comprising the gene encoded by (SEQ ID NO. 1) operably inked to an expression control sequence of a suitable host cell, wherein the host cell is preferably E. coli.

Another embodiment of the invention relates to method for manufacturing a fusion CA-CBD polypeptide comprising the steps of: transforming a suitable host cell with the isolated polynucleotide or a vector comprising the polynucleotide, culturing said cell under conditions allowing expression of said polynucleotide.

Yet another embodiment of the invention is a method of producing CA-CBD protein which comprises incorporating nucleic acids having the sequences provided by this invention into an expression vector, transforming a host cell with the vector and culturing the transformed host cell under conditions which result in expression of the gene.

Another embodiment of this invention is genetically engineered polypeptides created using the hybrid sequences of this invention.

Yet another aspect of this invention is utilizing the genetically engineered polypeptides created using the isolated nucleotide sequences of this invention.

Another embodiment of the invention relates to a method and system for removing CO₂ from a gas stream or other CO₂ rich environ using one of the invented fusion CA polypeptides.

In a preferred embodiment a fusion CA-CBD enzyme is utilized to remove CO₂ from a gas stream or other environ. One preferred method generally comprises: producing a medium, broth, lysate, or other mixture containing an amount of the invented fusion CA-CBD polypeptide, pouring the solution onto a cellulosic support to immobilize the CA-CBD polypeptide onto the cellulose support, and contacting the immobilized CA-CBD polypeptide with a CO₂ rich gas or gas stream.

Another embodiment relates to a method for manufacturing a fusion CA-CBD polypeptide comprising the steps of: transforming a suitable host cell with the isolated polynucleotide or a vector comprising the polynucleotide, culturing said cell under conditions allowing expression of said polynucleotide.

In an alternate embodiment of the invention envisions fusion proteins comprising at least one CBD (or functional equivalent thereof) fused with one or more polypeptide (i.e. proteins/enzymes etc.) capable of degrading and/or processing known environmental contaminants (i.e. CO₂, NOx etc.) or other compound(s). Some such fusion proteins may be capable of processing several contaminants simultaneously by incorporating multiple polypeptides capable of processing different contaminants. For example, it may be possible be to create a fusion polypeptide incorporating carbonic anydrase for the processing of CO₂ and another polypeptide capable of processing another contaminant such as NOx. It is envisioned that all of the embodiments of the present invention could be combined with prior art and future processes, methods, systems relating to processing of contaminants.

### BRIEF DESCRIPTION OF THE DRAWINGS

FIG. 1—is a restriction map of the pRSET plasmid

FIG. 2—is diagram of the pRSET-CA-CBD construct containing a His Tag a the N-terminal

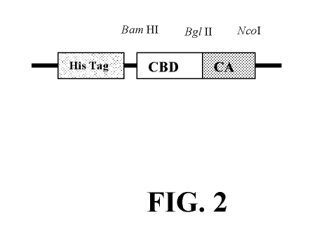

FIG. 3—is a diagram of one embodiment of the method for production and use of the CA-CBD hybrid peptide.

FIG. 4—SDS-PAGE gel comparing soluble and insoluble, induced and uninduced E. coli cells transformed with the CA-CBD hybrid gene, wherein Lanel is a Protein marker; Lane 2 is the soluble part of uninduced cells; Lane 3 is the soluble part of induced cells (0.5 mM IPTG); Lane 4 is the insoluble part of uninduced cells; Lane 5 is the insoluble part of induced cells (0.5 mM IPTG).

FIG. 5—illustrates binding isotherm for His-CBD-CA on PASC. Adsorption measurements of CBD-CA was performed in 50 mM phosphate buffer at pH8.0 and 22° C. [B] was the concentration of bound ligand (μmoles/g cellulose), [F] is the concentration of free ligand (μM).

### DETAILED DESCRIPTION OF THE PREFERRED EMBODIMENTS

In practicing the present invention several conventional techniques in microbiology and molecular biology (recombinant DNA) are used. Such techniques are well known and are explained in, for example, Sambrook, Molecular Cloning: A Laboratory Manual, Second Edition, Cold Spring Harbor Laboratory Press, Cold Spring Harbor, N.Y.; DNA Cloning: A practical Approach, 1985 (D. N. Glover ed); Current Protocols in Molecular Biology, John Wiley & Sons, Inc. (1994) and all more recent editions of these publications, all of which are hereby incorporated by reference in there entireties.

### DEFINITIONS

Before proceeding further with a description of the specific embodiments of the present invention, a number of terms will be defined.

As used herein, a “compound” or “molecule” is an organic or inorganic assembly of atoms of any size, and can include macromolecules, peptides, polypeptides, whole proteins, and polynucleotides.

As used herein, a “polynucleotide” is a nucleic acid of more than one nucleotide. A polynucleotide can be made up of multiple poly-nucleotide units that are referred to be a description of the unit. For example, a polynucleotide can comprise within its bounds a polynucleotide(s) having a coding sequence(s), a polynucleotide(s) that is a regulatory region(s) and/or other polynucleotide units commonly used in the art.

The “isolated nucleic acid” molecule of the present invention can include a deoxyribonucleic acid molecule (DNA), such as genomic DNA and complementary cDNA which can be single (coding or noncoding strand) or double stranded, as well as synthetic DNA, such as synthesized single stranded polynucleotide. The isolated nucleic acid molecule of the present invention can also include a ribonucleic acid molecule (RNA). Isolated nucleic acid is nucleic acid that is identified and separated from contaminant nucleic acid encoding other polypeptides from the source of nucleic acid. The nucleic acid may be labeled for diagnostic and probe purposes, using any label known and described in the art as useful in connection with diagnostic assays.

The determination of percent identity or homology between two sequences is accomplished using the algorithm of Karlin and Altschul (1990) Proc. Nat'l Acad. Sci. USA 87: 2264-2268, modified as in Karlin and Altschul (1993) Proc. Nat'l Acad. Sci. USA 90:5873-5877. Such an algorithm is incorporated into the NBLAST and XBLAST programs of Altschul et al. (1990) J. Mol. Biol. 215:403-410. BLAST nucleotide searches are performed with the NBLAST program, score=100, wordlength=12 to obtain nucleotide sequences homologous to the nucleic acid molecules of the invention. BLAST protein searches are performed with the XBLAST program, score=50, wordlength=3 to obtain amino acid sequences homologous to the protein molecules of the invention. To obtain gapped alignments for comparison purposes, Gapped BLAST is utilized as described in Altschul et al. (1997) Nucleic Acids Res. 25: 3389-3402. When utilizing BLAST and Gapped BLAST programs, the default parameters of the respective programs (e.g., XBLAST and NBLAST) are used. See the website for the national center for biological information.

As used herein, the terms hybridization (hybridizing) and specificity (specific for) in the context of nucleotide sequences are used interchangeably. The ability of two nucleotide sequences to hybridize to each other is based upon a degree of complementarity of the two nucleotide sequences, which in turn is based on the fraction of matched complementary nucleotide pairs. The more nucleotides in a given sequence that are complementary to another sequence, the greater the degree of hybridization of one to the other. The degree of hybridization also depends on the conditions of stringency, which include: temperature, solvent ratios, salt concentrations, and the like.

In particular, selective hybridization pertains to conditions in which the degree of hybridization of a polynucleotide of the invention to its target would require complete or nearly complete complementarity. The complementarity must be sufficiently high as to assure that the polynucleotide of the invention will bind specifically to the target relative to binding other nucleic acids present in the hybridization medium. With selective hybridization, complementarity will be 90-100%, preferably 95-100%, more preferably 100%.

The term stringent conditions is known in the art from standard protocols (e.g. Current Protocols in Molecular Biology, editors F. Ausubel et al., John Wiley and Sons, Inc. 1994) and is when hybridization to a filter-bound DNA in 0.5M NaHPO₄ (pH7.2), 7% sodium dodecyl sulfate (SDS), 1 mM EDTA at +65° C., and washing in 0.1×SSC/0.1% SDS at +68° C. is performed.

“Degenerate variant” refers to the redundancy or degeneracy of the genetic code as is well known in the art. Thus the nucleic acid sequences shown in the sequence listing provided only examples within a larger group of nucleic acids sequences that encode for the polypeptide desired.

Because the genetic code is degenerate, more than one codon may be used to encode a particular amino acid, and therefore, the amino acid sequence can be encoded by any set of similar DNA oligonucleotides. With respect to nucleotides, therefore, the term derivative(s) is also intended to encompass those DNA sequences that contain alternative codons which code for the eventual translation of the identical amino acid.

The term “vector” is used to refer to any molecule, including but not limited to nucleic acids, plasmids, or viruses used to transfer nucleic coding information to a host cell. One preferred of vector is a “plasmid”, which refers to a circular double stranded DNA loop into which additional DNA segments can be ligated. Certain vectors are capable of directing the expression of genes to which they are operatively linked. Such vectors are referred to herein as “expression vectors”. In general, expression vectors of utility in recombinant DNA techniques are often in the form of plasmids. Expression includes, but is not limited to, processes such as transcription, translation, and RNA splicing, if introns are present. The terms “plasmid” and “vector” can be used interchangeably herein. However, the invention is intended to include such other forms of expression vectors, such as viral vectors.

The term “host cell” refers to a cell which is capable of being transformed with a nucleic acid sequence and then of expressing a selected gene of interest. The term host cell includes the progeny of the parent cell.

As used herein, the terms “transformation” and “transfection” are intended to refer to techniques known in the art for introducing foreign nucleic acid into a host cell. Suitable methods for transforming or transfecting host cells can be found in Sambrook, et al. (Molecular Cloning: A Laboratory Manual, 2.sup.nd, ed. Cold Spring Harbor Laboratory, Cold Spring Harbor Laboratory Press, Cold Spring Harbor, N.Y., 1989) and other laboratory manuals. Following transfection or transduction, the transforming DNA may recombine with that of the cell by physically integrating into a chromosome of the cell, may be maintained transiently as an episomal element without being replicated, or may replicate independently as a plasmid. A cell is considered to have been stably transformed when the DNA is replicated with the division of the cell.

A “transformed cell” is a cell in which a nucleic acid (of the invented nucleic acids described herein) has been inserted by means of recombinant DNA techniques. A host cell can be chosen that modifies the expression of the inserted sequence(s), or modifies and processes the gene product in a specific, desired fashion. Modifications such as glycosylation and processing likes cleavage of protein products may facilitate optimal function of the protein.

The term “native” when used in connection with biological materials such as nucleic acid molecules, polypeptides, etc. . . . refers to materials which are found in nature and which have not been manipulated by humans. “Non-native” as used herein refers to a material that is not found in nature or that has been structurally modified or synthesized by humans.

The terms “peptide” is used to indicate a chain of at least two amino acids coupled by peptide linkages. The word “polypeptide” is used herein for chains containing more than ten amino acid residues

“Isolated” polypeptide or protein is intended a polypeptide or protein removed from its native environment.

“operatively linked” is intended to mean that the nucleotide sequence of interest is linked to the regulatory sequence(s) in a manner which allows for expression of the nucleotide sequence. A coding sequence is operably linked to a promoter when the promoter is capable of directing transcription of that coding sequence.

The term “regulatory sequence” is intended to include promoters, enhancers and other expression control. Regulatory sequences include those which direct constitutive or inducible expression of a nucleotide sequence in many host cells.

“Biologically active fragments” of a polypeptide of the invention include polypeptides comprising amino acid sequences sufficiently identical to or derived from the amino acid sequence of the CA-CBD fusion which include fewer amino acids than the full length protein, and exhibit at least a significant amount of the carbonic anydrase and CBD properties of the corresponding full-length protein. A biologically active fragment of a protein of the invention can be a polypeptide which is, for example, 10, 25, 50, 100 or more amino acids in length. Moreover, other biologically active portions, in which other regions of the protein are deleted, can be prepared by recombinant techniques and evaluated for one or more of the biological activities of the native form of a polypeptide of the invention. Embodiments of the invention also features nucleic acid fragments which encode the above biologically active fragments of the CA-CBD enzyme protein.

The terms “functional equivalents” and “functional variants” are used interchangeably herein. Functional equivalents of the CA-CBD enzyme encoding DNA are isolated DNA fragments that encode a polypeptide that exhibits the carbonic anhydrase and cellulose binding functions of the CA-CBD polypeptide. A functional equivalent of a CA-CBD enzyme polypeptide according to the invention is a polypeptide that exhibits both above-identified functions of the CA-CBD enzyme as defined herein. Functional equivalents therefore also encompass biologically active fragments.

Chemical equivalency can be determined by one or more the following characteristics: charge, size, hydrophobicity/hydrophilicity, cyclic/non-cyclic, aromatic/non-aromatic etc. For example, a codon encoding a neutral non-polar amino acid can be substituted with another codon that encodes a neutral non-polar amino acid, with a reasonable expectation of producing a biologically equivalent protein.

Amino acids can generally be classified into four groups. Acidic residues are hydrophilic and have a negative charge to loss of H⁺ at physiological pH. Basic residues are also hydrophilic but have a positive charge to association with H⁺ at physiological pH. Neutral nonpolar residues are hydrophobic and are not charged at physiological pH. Neutral polar residues are hydrophilic and are not charged at physiological pH. Amino acid residues can be further classified as cyclic or noncyclic and aromatic or nonaromatic, self-explanatory classifications with respect to side chain substituent groups of the residues, and as small or large. The residue is considered small if it contains a total of 4 carbon atoms or less, inclusive of the carboxyl carbon. Small residues are always non-aromatic.

Of naturally occurring amino acids, aspartic acid and glutamic acid are acidic; arginine and lysine are basic and noncylclic; histidine is basic and cyclic; glycine, serine and cysteine are neutral, polar and small; alanine is neutral, nonpolar and small; threonine, asparagine and glutamine are neutral, polar, large and nonaromatic; tyrosine is neutral, polar, large and aromatic; valine, isoleucine, leucine and methionine are neutral, nonpolar, large and nonaromatic; and phenylalanine and tryptophan are neutral, nonpolar, large and aromatic. Proline, although technically neutral, nonpolar, large, cyclic and nonaromatic is a special case due to its known effects on secondary conformation of peptide chains, and is not, therefore included in this defined group. There are also common amino acids which are not encoded by the genetic code include by example and not limitation: sarcosine, beta-alanine, 2,3-diamino propionic and alpha-aminisobutryric acid which are neutral, nonpolar and small; t-butylalanine, t-butylglycine, methylisoleucine, norleucine and cyclohexylalanine which are neutral, nonpolar, large and nonaromatic; ornithine which is basic and non-cylclic; cysteic acid which is acidic; citrulline, acetyl lysine and methionine sulfoxide which are neutral, polar, large and nonaromatic; and phenylglycine, 2-naphtylalanine, B-2-thienylalanine and 1,2,3,4-tetrahydroisoquinoline-3-carboxylic acid which are neutral, nonpolar, large and aromatic. Other modifications are known in the art some of which are discussed in U.S. Pat. No. 6,465,237 issued to Tomlinson on Oct. 15, 2002.

### Approach for Production of Novel CA-CBD Protein

The present inventors have recently investigated the energy-efficient capture of CO₂ using biologically derived enzymes. One of the largest hurdles to the large scale use of such enzymes is the cost and difficulty associated with enzyme immobilization.

The inventors have developed a method and system for the energy-efficient capture of CO₂ by employing a hybrid protein comprising the enzyme carbonic anhydrase (CA) which efficiently catalyzes the hydration of CO₂, fused to a cellulose-binding domain (CBD) that would allow the protein to bind to cellulose. This novel bifunctional protein can bind tightly to cellulose while maintaining its native catalytic activity in relations to CO₂. The ability of the hybrid protein to efficiently bind to cellulose is important because cellulose is a superb matrix for enzyme immobilization due to its low cost and exceptional physical properties

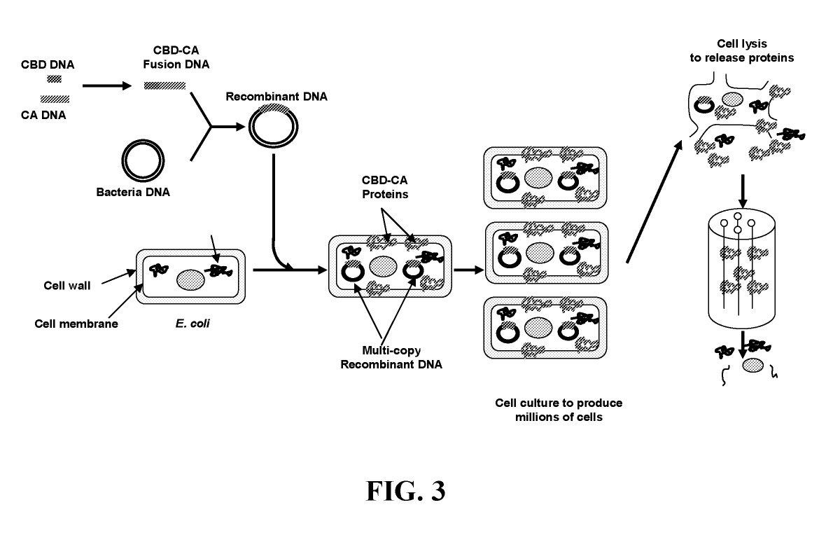

FIG. 3 illustrates one general approach of producing the fusion CA-CBD protein comprising cloning relevant DNA sequences encoding CBD and a CA into an expression vector, transforming a suitable host with the vector containing the hybrid CA-CBD gene, cultivating the host and inducing expression of the fusion protein, and recovering and/or using the expressed CA-CBD protein. Initial research focused on selecting the appropriate CBDs and CAs and expressing the CA-CBD hybrid gene in a host for the production of the novel CA-CBD protein.

### Cellulose Binding Domain

A number of enzymes that catalyze cellulose degradation contain cellulose binding domains (CBDs) that allow the protein to bind tightly to the cellulose fiber during the catalytic hydrolysis of cellulose. Cellulose binding domain (CBD) from Bacillus licheniformis and Clostridium cellulovorans were initially selected for fusion to CA do to their high affinity for cellulose. However, the use of the cellulose binding domain (CBD) from Clostridium cellulovorans led to formation of CA-CBD almost entirely in inclusion bodies being expressed in E. coli which makes it a possible put less preferable option.

Examination of two other CBDs, one from Clostridium cellulolyticum and the other from Clostridium thermocellum were also investigated since they display high affinity towards cellulose but unlike CBD from Clostridium cellulovorans they are produced in soluble form in E. coli. While a variety of CBD may be employed the CBD, Clostridium thermocellum is a preferred CBD, especially when utilizing an E. coli host.

### Carbonic Anhydrase

Photosynthetic organisms have been effectively trapping atmospheric CO₂ for billions of years. This biological trapping system is most effective with CO₂ that is in the hydrated (carbonate ion) form. In biological systems protein catalysts have evolved to perform this hydration of CO₂. The enzyme carbonic anhydrase catalyzes the reaction: CO₂+H₂O [symbol] (HCO₃⁻+H⁺. In fact, carbonic anhydrase is one of the most active known enzymes with a turnover rate (the number of reactions at a single active site) of 10⁶/sec. This translates to the hydration of 1.3 grams of CO₂ in one second by just 1 milligram of enzyme or, 4.68 million kilograms/hour using just one kilogram of enzyme under ideal conditions. The carbonic anhydrase from Neisseria gonorrhoeae was selected for initial experiments. It hydrates CO₂ at rates as high as kcat=1.1*106 s−1, or a million times a second. The nonenzymatic reaction rate is 1.3*10−1 s−1. Thus the rate enhancement achieved by using the carbonic anhydrase from Neisseria gonorrhoeae is about 8.5*106. Although initial research focused on using the full length CA of N. gonorrhoeae it was found that the use of a mature version of the protein was preferable.

Although CA of N. gonorrhoeae is preferable other CBDs may also be employed. Production of a hybrid CA-CBD gene and production of the CA-CBD protein encoded by such a hybrid gene is discussed in detail below.

### Preferred Embodiments

The present invention generally relates to a fusion carbonic anhydrase with enhanced cellulose binding. One embodiment of the invention relates to a fusion CA-CBD polypeptide, the nucleotide sequences encoding such a protein, and a method of use and production thereof. The present invention is particularly well suited for catalyzing/removing carbon dioxide from a gas stream or other environment containing carbon dioxide. The invented bi-functional protein allows one to immobilize CA with inexpensive cellulose supports making the use of CA for processing CO₂ much more economically viable.

### Polypeptide Sequence

One embodiment of the invention generally relates to a fusion polypeptide comprising: a carbonic anhydrase or carbonic anhydrase variant fused with a heterologous amino acid sequence, wherein the heterologous amino acid sequence is a polypeptide that aids in the attachment of the carbonic anhydrase to cellulose.

The invention provides an isolated polypeptide having the amino acid sequence selected from the group consisting of (SEQ ID NO. 8 and SEQ ID NO. 14) and amino acid sequences obtainable by expressing the polynucleotide selected from the group consisting of (SEQ ID NO. 1; SEQ ID NO. 12; and SEQ ID NO. 13) in an appropriate host. The invention contemplates certain modifications to these sequence, including deletions, insertions, and substitutions, that are well known to those skilled in the art as well as functional equivalents thereof.

Polypeptides of the present invention include naturally purified products, products of chemical synthetic procedures, and products produced by recombinant techniques from a prokaryotic or eukaryotic host, including, for example, bacterial, yeast, higher plant, insect and mammalian cells. Depending upon the host employed in a recombinant production procedure, the polypeptides of the present invention may be glycosylated or may be non-glycosylated.

The above polypeptides are collectively comprised in the term “polypeptides according to the invention.

### Nucleotide Sequences

The scope of the present invention is not limited to the exact sequence of the nucleotide sequences set forth in (SEQ ID NO: 1) or the use thereof. The invention contemplates certain modifications to the sequence, including deletions, insertions, and substitutions, that are well known to those skilled in the art as well as functional equivalents thereof.

For example, the invention contemplates modifications to the sequence found in (SEQ ID NO: 1) with codons that encode amino acids that are chemically equivalent to the amino acids in the native protein. An amino acid substitution involving the substitution of amino acid with a chemically equivalent amino acid is known as a conserved amino acid substitution.

Exemplary nucleotide sequences encoding the novel CA-CBD include but are not limited to: (SEQ ID NO.1; SEQ ID NO. 12; and SEQ ID NO. 13) and functional equivalents thereof.

### Vectors and Host Cells

Another embodiment of the invention pertains to vectors, preferably expression vectors, containing at least a nucleic acid encoding a protein according to the invention or a functional/chemical equivalent thereof.

The expression vectors of the invention comprise a nucleic acid of the invention in a form suitable for expression of the nucleic acid in a host cell. Various vectors can be employed as long as the expression vector includes one or more regulatory sequences, selected on the basis of the host cells to be used for expression, which is operatively linked to the nucleic acid sequence to be expressed.

The recombinant expression vectors of the invention are preferably designed for expression of CA-CBD fusion enzymes in prokaryotes, however, it may be possible to design them to be expressed in eukaryotic cells as well. Preferably the protein according to the invention is expressed in bacterial cells such as E. coli and Bacillus species.

It may also be possible to express the enzyme in other system such as insect cells, yeast cells or mammalian cells. Alternatively, the recombinant expression vector can be transcribed and translated in vitro, for example using T7 promoter regulatory sequences and T7 polymerase. The DNA insert should be operatively linked to an appropriate promoter, such as the E. coli lac, trp and tac promoters, or other suitable promoters known in the art Vector DNA can be introduced into prokaryotic or eukaryotic cells via conventional transformation or transfection techniques.

Exemplary vectors include vectors having the sequence of (SEQ ID NO. 2; SEQ ID NO. 18; SEQ ID NO. 19) and functional equivalents thereof.

### Polypeptide Production

Host cells comprising a CA-CBD polypeptide expression vector may be cultured using standard media well known to the skilled artisan. Standard media will usually contain all nutrients necessary for the growth and survival of the cells. Suitable media for culturing E. coli cells include, for example, Luria Broth (LB) and/or Terrific Broth (TB). Suitable media for culturing eukaryotic cells are also known in the art.

Typically, an antibiotic or other compound useful for selective growth of transfected or transformed cells is added as a supplement to the media. The compound to be used will be dictated by the selectable marker element present on the plasmid with which the host cell was transformed. For example, where the selectable marker element is ampicillin resistance, the compound added to the culture medium will be ampicillin. Other compounds for selective growth include kanamycin, tetracycline, and neomycin.

The amount of a CA-CBD polypeptide produced by a host cell can be evaluated using standard methods known in the art. Such methods include, without limitation, Western blot analysis, SDS-polyacrylamide gel electrophoresis, non-denaturing gel electrophoresis, High Performance Liquid Chromatography (HPLC) separation, immunoprecipitation, and/or activity assays such as DNA binding gel shift assays.

For a CA-CBD polypeptide situated in the host cell cytoplasm and/or nucleus (for eukaryotic host cells) or in the cytosol (for bacterial host cells), the intracellular material (including inclusion bodies for gram-negative bacteria) can be extracted from the host cell using any standard technique known to the skilled artisan. For example, the host cells can be lysed to release the contents of the periplasm/cytoplasm by French press, homogenization, and/or sonication followed by centrifugation.

If a CA-CBD polypeptide has been designed to be secreted from the host cells, the majority of polypeptide may be found in the cell culture medium. If however, the CA-CBD polypeptide is not secreted from the host cells, it will be present in the cytoplasm and/or the nucleus (for eukaryotic host cells) or in the cytosol (for gram-negative bacteria host cells).

### Purification

Since the CA-CBD polypeptide can easily be immobilized on a cellulose support by pouring or otherwise applying the lyste and/or other solution containing the expressed polypeptide onto a cellulose support, a purification step is unnecessary as the solution containing the free polypeptide can be poured or otherwise immobilized directly onto a cellulose support.

Although if a purified fusion protein is desired, purification of a CA-CBD polypeptide from solution can be accomplished using a variety of techniques. If the polypeptide has been synthesized such that it contains a tag such as Hexahistidine (CA-CBD polypeptide/hexaHis) or other small peptide such as FLAG (Eastman Kodak Co., New Haven, Conn.) or myc (Invitrogen, Carlsbad, Calif.) at either its carboxyl or amino-terminus, it may be purified in a one-step process by passing the solution through an affinity column where the column matrix has a high affinity for the tag.

For example, polyhistidine binds with great affinity and specificity to nickel. Thus, a nickel affinity column can be used for purification of CA-CBD polypeptide/polyHis. See, e.g., Current Protocols in Molecular Biology .sctn. 10.11.8 (Ausubel et al., eds., Green Publishers Inc. and Wiley and Sons 1993).

In situations where it is preferable to partially or completely purify a CA-CBD polypeptide such that it is partially or substantially free of contaminants, standard methods known to those skilled in the art may be used. Such methods include, without limitation, separation by electrophoresis followed by electroelution, various types of chromatography (affinity, immunoaffinity, molecular sieve, and ion exchange), HPLC, and preparative isoelectric focusing (“Isoprime” machine/technique, Hoefer Scientific, San Francisco, Calif.). In some cases, two or more purification techniques may be combined to achieve increased purity.

CA-CBD polypeptides may also be prepared by chemical synthesis methods (such as solid phase peptide synthesis) using techniques known in the art such as those set forth by Merrifield et al., 1963, J. Am. Chem. Soc. 85:2149; Houghten et al., 1985, Proc Natl Acad. Sci. USA 82:5132. U.S. Pat. Nos. 5,763,192, 5,814,476 and others, describe various exemplary processes for producing peptides or polypeptides which are hereby incorporated by reference in their entireties.

### Example I PCR Amplification and Purification of CA DNA

N. gonorrhoeae genomic CA DNA was obtained from ATTC (ATTC No. 53422D; SEQ ID NO: 10) and used as a target for PCR reactions to amplify the CA DNA. The DNA used encodes a mature carbonic anhydrase which lacks 25 amino acids of signal peptide. Although a number of CAs may be employed, mature CA DNA was found to produce a hybrid CA-CBD protein that was much easier to refold and avoided inclusion body problems encountered with the use of several other CA constructs.

The PCR primers shown in Table I were designed to provide specific-restriction sites at the ends so that the amplified product could be cloned directly into the vector plasmid into plasmid pRSET-B. The plasmid carrying an integrated CA is referred to as R1.

TABLE I
Primers for PCR of CA

| | | | DNA Sequence: | | Restriction |
|---|---|---|---|---|---|
| Primer | F/R | Target | 5′ to 3′ | nt | Site |
| CAf | F | CA | ATTTGCAGATCTCAC | 30 | Bgl II |
| | | | GGCAATCACACCCA | | |
| | | | SEQ ID NO. 5 | | |
| Car | R | CA | ACGGccatggTTATT | 29 | Nco I |
| | | | CAATAACTACACGT | | |
| | | | SEQ ID NO. 6 | | |

PCR amplification was achieved by incubating the PCR reaction mixture in a thermal cycler at 94° C. for 5 min to completely denature the template and activate the enzyme. Performed 30 cycles of PCR amplification as follows: Denature at 94° C. for 30 sec, anneal at 50° C. for 30 sec, extend at 72° C. for 1 min followed by an additional extension at 72° C. for 10 min. The content was kept at 4° C. A detailed protocol is further explained in Sambrook, Molecular Cloning: A Laboratory Manual, Second Edition, Cold Spring Harbor Laboratory Press, Cold Spring Harbor, N.Y.; DNA Cloning: A practical Approach.

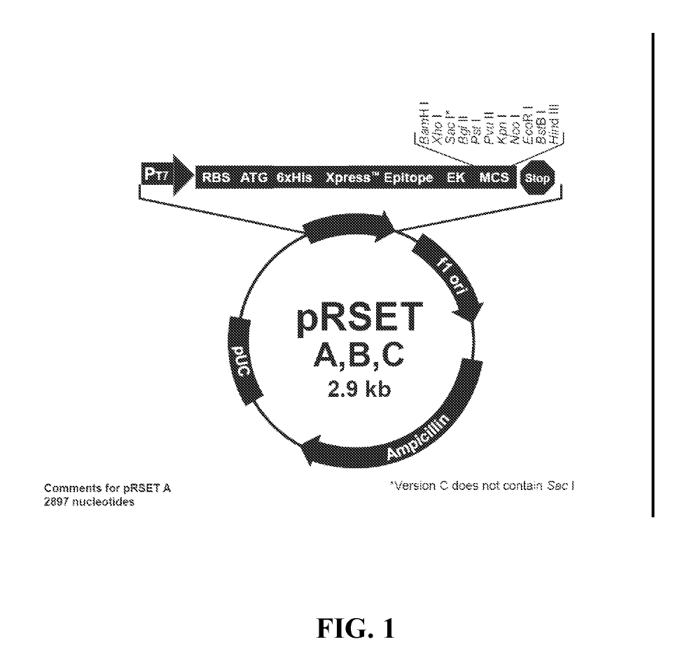

The amplified CA DNA was then digested with Bgl II/Nco I, purified again and ligated with the recombinant plasmid pRSET B (See, FIG. 1) which has been digested with the same enzymes. The restriction enzymes were obtained from (New England Biolabs, Ipswich, Mass.). Reaction: Relevant DNA 1 μg, Bgl II 1 μl and Nco I 1, BSA 2 μl, restriction buffer 2 μl, were mixed and total volume increased to 20 μl. The mixture was incubated at 37° C. for 1 h. After running the DNA fragments were purified by QIAEX® II Gel Extraction Kit manufactured by Qiagen (Alameda, Calif.).

The ligation was performed at 4° C. overnight. Reaction: Restricted plasmid 50 ng, restricted DNA fragment 100 ng, T4 ligation buffer 1 μl, 5 mM ATP 1 μl, ligase 0.5 μl, were mixed in a total volume of 10 μl.

### Example II Construction of Plasmid R2 Carrying CA-CBD Fusion

DNA from Clostridium thermocellum was obtained from ATTC (ATTC No. 27405D) and used as a target for PCR reactions to amplify the CBD gene. The PCR primers shown in Table II. PCR amplification was achieved by incubating the PCR reaction mixture in a thermal cycler at 94° C. for 5 min to completely denature the template and activate the enzyme. Performed 30 cycles of PCR amplification as follows: Denature at 94° C. for 30 sec, anneal at 50° C. for 30 sec, extend at 72° C. for 1 min followed by an additional extension at 72° C. for 10 min. The content was kept at 4° C. A detailed RT-PCR protocol is further explained in Sambrook, Molecular Cloning: A Laboratory Manual, Second Edition, Cold Spring Harbor Laboratory Press, Cold Spring Harbor, N.Y.; DNA Cloning: A practical Approach.

The amplified CBD DNA were digested by Bam HI/Bgl II and then ligated to Plasmid pRSET-R1 which was also digested with the same enzymes to create plasmid R2 (i.e. pRSET-CBD-CA)

The restriction enzymes were obtained from (New England Biolabs, Ipswich, Mass.). Reaction: Relevant DNA 1 μg, Bam HI and Bgl II, BSA 2 μl, restriction buffer 2 μl, were mixed and total volume increased to 20 μl. The mixture was incubated at 37° C. for 1 h. After running the agarose gel DNA fragments were purified by QIAEX® II Gel Extraction Kit manufactured by Qiagen (Alameda, Calif.).

The ligation was performed at 4° C. overnight. Restricted plasmid 50 ng, restricted DNA fragment 100 ng, T4 ligation buffer 1 μl, 5 mM ATP 1 μl, ligase 0.5 μl, were mixed in a total volume of 10 μl.

The ligated product was used to transform competent E. coli (BL21 (DE3) pLysS) by mixing ligation product with the E. coli on ice for 30 min in a tube, (See, Molecular Cloning: A Laboratory Manual, 2.sup.nd, ed. Cold Spring Harbor Laboratory, Cold Spring Harbor Laboratory Press, Cold Spring Harbor, N.Y., 1989-Protocol II: Fresh competent E. coli prepared using calcium chloride as described in pages 1.82 to 1.84 of the above reference.) The tube was transferred to a 42° C. water for 60 seconds. The tube was then moved back to a bucket of ice and kept for 90 seconds. The contents were then poured onto LB medium containing 50 ug/ml ampicillin. A positive colony was selected and R2 was identified by PCR and enzyme digestion and sequencing. The clones were selected from the relevant antibiotic plates. The plasmid was extracted and the enzyme digestion was performed for the selection of the positive according to the size of insert. The PCR amplification was also used for the selection according to the size of amplified PCR product. DNA Sequence was performed by Sanger sequencing method.

The DNA sequence for the pRSET-CBD-CA plasmid was determined to the sequence in SEQ ID NO: 2 encoding the amino acid sequence SEQ ID NO: 9.

TABLE II
Primers for PCR of CBD

| | | | | | |
|---|---|---|---|---|---|
| | | | DNA Sequence: | | Restriction |
| Primer | F/R | Target | 5′ to 3′ | nt | Site |
| CBDf | F | CBD | GCGGGATCCGGAATT | 3 | Bam HI |
| | | | CTACAACAGCAATCC | 0 | |
| | | | SEQ ID NO: 3 | | |
| CBDr | R | CBD | ATTTGCAGATCTATC | 2 | Bgl II |
| | | | ATCTGACGGCGGT | 8 | |
| | | | SEQ ID NO: 4 | | |

### Example III Identification of CBD-CA

The ligated-CBD-CA fusion was used to transform competent E. coli cells which were placed in LB medium containing 50 ug/ml ampicillin. A colony was selected and the recombinant plasmid R2 carrying the CA-CBD fusion was identified by PCR, enzyme digestion and DNA sequencing. The clones were selected from the relevant antibiotic plates. The plasmid was extracted and the enzyme digestion was performed for the selection of the positive according to the size of insert. The PCR amplification was also used for the selection according to the size of amplified PCR product and the product was sequenced. DNA Sequence was performed by Sanger sequencing method.

The DNA sequence for the CBD-CA was determined to the sequence in SEQ ID NO: 1 encoding the amino acid sequence SEQ ID NO: 8.

### Example IV Transformation of E. coli Host and Expression of Host Protein

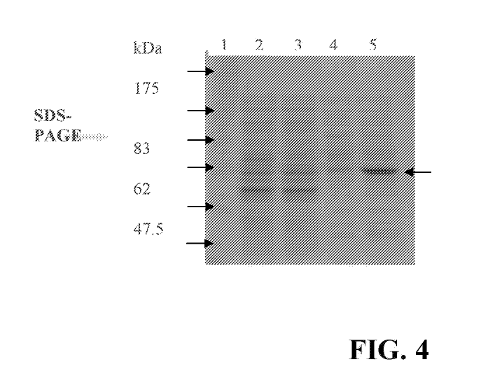

The recombinant vector R2 containing CBD and CA was transformed to BL21 (DE3) pLysS as the host strain (See, Molecular Cloning: A Laboratory Manual, 2.sup.nd, ed. Cold Spring Harbor Laboratory, Cold Spring Harbor Laboratory Press, Cold Spring Harbor, N.Y., 1989-Protocol II: Fresh competent E. coli prepared using calcium chloride as described in pages 1.82 to 1.84 of the above reference.) A well-grown single colony from the selection plate was inoculated into culture medium supplemented with 50 ug/mL ampicillin overnight at 37° C. 10 mL of the culture was added to 1 L fresh culture medium and the culture was grown in an orbital shaker at 37° C. for 2-3 h, to OD₆₀₀ of 0.6. Thereafter, expression of CBD-CA was induced by adding 0.5 mM concentration of lactose and incubation was continued for 8-10 h at 25° C. The expression system produced high levels of CA-CBD reaching 8% of total cell protein at 8 h post induction. See, FIG. 4.

### Example V Purification Protocol of CBD-CA

E coli cells were collected by centrifugation and resuspended in 10 ml of B-PER® II Bacterial Protein Extraction Reagent (Pierce, Rockford, Ill.) Prod#78260, containing 20 μl 100 mg/ml lysozyme, 200 μl Triton X-100, 20 μl Dnase I buffer, 50 μl Dnase I, 100 μl 100 mM MgCl2, and 5 μl 100 mM PMSF. After being incubated on ice for 30 min, the suspension was subjected to ultrasonication for 3 min at 30% of maximum amplitude using a Vibra cell ultrasonifier (Fisher Bioblock Scientific, Illkirch, France). The lysate was centrifuged for 10 min at 15,000×g to remove cell debris and then applied onto 4 ml of nickel ProBond™ resin (Invitrogen, Carlsbad, Calif.), and the proteins bound to the resin were purified and pooled according to the product manual. The pooled solution was dialyzed with 200 ml 20 mM NaH2PO4, 500 mM NaCl (pH8.0) twice for 36 h, and concentrated using Amicon® Ultra 30-kDa membrane (Millipore, Billerica, Mass.).

### Example VI Inclusion Bodies

Early attempts to fuse the CBD of Clostridium cellulovorans to the full length carbonic anhydrase of N. gonorrhoeae did not yield protein. Three different kinds of plasmids, PUC19, pET-CBD 180, and pET20 b(+) were used for the expression vectors and isopropyl thio-β-D-galactoside (IPTG) as the inducer. The recombinant plasmids were transformed into E. coli TOP10 and the positive clones were transformed to E. coli BL21. Unfortunately, the fusion genes did not express.

It was found that using mature CA (without a 26 amino acids signal peptide), the fusion was produced as inclusion bodies using pET-CBD 180 or pET20 b(+). However, the yield of active protein after denaturing and refolding of the inclusion bodies was very low (around 5% in the total inclusion bodies). Next, CBD of C. cellulovorans was substituted for that of C. thermocellum, the fusion protein was expressed as soluble protein but the yield was still low.

Finally, we chose the pREST-B as expression vector, BL21 (DE3)pLysS as host strain, and lactose as inducer. This expression system produced high levels of BD-CA reaching 8% of the total cell protein at about 8 h post-induction.

### Inclusion Bodies

If a CBD-CA polypeptide has formed inclusion bodies in the cytosol, the inclusion bodies can often bind to the inner and/or outer cellular membranes and thus will be found primarily in the pellet material after centrifugation. The pellet material can then be treated at pH extremes or with a chaotropic agent such as a detergent, guanidine, guanidine derivatives, urea, or urea derivatives in the presence of a reducing agent such as dithiothreitol at alkaline pH or tris carboxyethyl phosphine at acid pH to release, break apart, and solubilize the inclusion bodies. The solubilized CBD-CA polypeptide can then be analyzed using gel electrophoresis, immunoprecipitation, or the like. If it is desired to isolate the CBD-CA polypeptide, isolation may be accomplished using standard methods such as those described herein and in Marston et al., 1990, Meth. Enz., 182:264 75.

Other methods known in the art for “refolding” or converting the polypeptide to its tertiary structure and generating disulfide linkages can be used to restore biological activity. Such methods include but are not limited to exposing the solubilized polypeptide to a pH usually above 7 and in the presence of a particular concentration of a chaotrope. The selection of chaotrope is very similar to the choices used for inclusion body solubilization, but usually the chaotrope is used at a lower concentration and is not necessarily the same as chaotropes used for the solubilization. In most cases the refolding/oxidation solution will also contain a reducing agent or the reducing agent plus its oxidized form in a specific ratio to generate a particular redox potential allowing for disulfide shuffling occurring in the formation of the protein's cysteine bridges. Some of the commonly used redox couples include cysteine/cystamine, glutathione (GSH)/dithiobis GSH, cupric chloride, dithiothreitol(DTT)/dithiane DTT, and 2-2-mercaptoethanol(bME)/dithio-b(ME). In many instances, a co-solvent may be used or may be needed to increase the efficiency of the refolding, and the more common reagents used for this purpose include glycerol, polyethylene glycol of various molecular weights, arginine and the like.

If inclusion bodies are not formed to a significant degree upon expression of a CBD-CA polypeptide, then the polypeptide will be found primarily in the supernatant after centrifugation of the cell homogenate.

### Example VII Identification of Clones

The clones were selected from the relevant antibiotic plates. The plasmid was extracted and the enzyme digestion was performed for the selection of the positive according to the size of insert. The PCR amplification was also used for the selection according to the size of amplified PCR product.

Method and System for Removing CO₂ from a Gas or Gas Stream

Another embodiment of the invention relates to a method and system for removing CO₂ from a gas stream. One preferred embodiment generally comprises:
a. providing a fusion polypeptide with carbonic anhydrase activity and the ability to bind to cellulose;
b. immobilizing the fusion polypeptide onto a cellulose containing support forming an activated support;
c. contacting the activated support with a CO₂ containing gas or gas stream, wherein the fusion polypeptide reacts with at least a portion of the CO₂ in the gas or gas stream forming carbonate ions, bicarbonate ions, carbonate, bicarbonate or combinations thereof.

The method preferably employs a fusion polypeptide of the type described herein, or a derivative, fragment and/or equivalent thereof. The fusion polypeptide is immobilized on to a cellulose or cellulose containing support. A variety of cellulose, lignin-cellulose, cellulose containing, and cellulose derivative supports known in the art can be employed.

The fusion polypeptide is easily immobilized onto the cellulose support by pouring onto, immersing in or otherwise applying a lysate, broth, or other solution containing the fusion polypeptide to the cellulose support. The cellulose binding domains for the fusion polypeptide provide a binding means for the fusion polypeptide to attach to the cellulose support.

Once immobilized, the immobilized fusion polypeptide can be contacted with a CO₂ containing gas to convert the CO₂ into CO₂ reaction production including but not limited to: carbonate ions, bicarbonate ions, carbonate, bicarbonate or combinations thereof. The immobilized fusion protein can be placed in to a variety of reactors including but not limited to basket reactors and absorption columns to effectively implement the immobilized fusion protein for use in power plants and other CO₂ rich environs.

The resulting catalyzed CO₂ reaction products (i.e. bicarbonate ions) can be stabilized by in several ways. The reaction products can be contacted with calcium and/or magnesium-bearing solutions to promote mineralization, they can be re-evolved in a CO₂ concentrated sweep stream through concentration gradient and compressed to a liquid and shipped to a long-term sequestration destination. Alternatively, they can be converted, via other biological processes, to mineral carbonates or fixed as simple organics.

### Method for Producing a Fusion CA-CBD Polypeptide

One embodiment of the invention relates to a method for manufacturing a fusion CA-CBD polypeptide comprising the steps of: transforming a suitable host cell with the isolated polynucleotide or a vector comprising the polynucleotide, culturing said cell under conditions allowing expression of said polynucleotide, details of which are described herein.

### Characterization of CA-CBD

(1) Electrometric assay for CA activity—The electrometric method in which the time required (in seconds) for a saturated CO₂ solution to lower the pH of 0.012 M Tris SO₄ buffer from 8.3 to 6.3 at 0° C. was also used with some modifications. A 10 μl portion of cell extract or purified proteins was diluted into a final volume of 6 ml of prechilled 20 mM Tris SO₄ buffer, pH8.3. The mixture was stirred and maintained on ice for several minutes. The assay was initiated by the addition of 4 ml of ice-cold, CO₂-saturated water into the reaction vessel. The change in pH from 8.3 to 6.3 was monitored using a pH meter. CA activity was described in Wilbur-Anderson (WA) units per mg of protein and was calculated using the formula [2*(T₀−T)/T]/mg protein, where T₀ and T represent the time required for the pH to change from 8.3 to 6.3 in control and cell extract buffers, respectively.

Results: Crude cell extracts of pRET-CBD-CA clones were used for measuring CA activities. CA activities was detected in cell extracts of induced pRET-CBD-CA clones but not in those of uninduced clones (Table III). And the crude had the specific activities of 8.02 WA/mg. The fusion proteins purified from pRET-CBD-CA had the specific activities of 100 WA/mg. The fusion proteins were treated with thrombin protease to cleave the His tag, and the enzyme activities of the native and the His-tagged proteins were determined. The His-tagged and native enzymes were equally active, showing that the His-tag did not affect activity. Therefore, all further experiments were carried out with the His-tagged recombinant proteins.

TABLE III
Purification of His-CBD-CA

| | Total | | Specific | Specific | purification |
|---|---|---|---|---|---|
| Sample | activity | Protein | Activity | Activity | fold |
| Unit of a Activity | WA^{a} | mg^{b} | WA/mg | WA/μmol | — |
| or mass | | | | | |
| Cell extract | 682 | 85 | 8.02 | — | 1 |
| After partial | 500 | 5 | 100 | 5000 | 12 |
| purification using | | | | | |
| Ni (II) column | | | | | |

^{a}One WA = [2 * (T₀ − T)/T]/mg protein, where T₀ and T represent the time required for the pH to change from 8.3 to 6.3 in control and cell extract buffers, respectively.
^{b}Determined by BCA protein assay.

(2) Binding isotherm measurements—All adsorption-isotherm measurements were carried out at 22° C. in 1.5 ml Eppendorf tubes. The samples containing 1-300 μM of His-CBD-CA and 1% bovine serum albumin (BSA) mixed with 0.5 mg of phosphoric acid-swollen cellulose (PASC) in 50 mM NaH₂PO₄, 500 mM NaCl pH8.0 buffer, to a final aqueous volume of 1.0 ml. Control tubes contained no PASC. Each solution was vortexed for 5 sec and then placed in a shaker for 1 h to allow equilibration. The samples were centrifuged at 4° C. and 10,000 rpm for 10 min to remove the protein-covered cellulose. The clear supernatant was collected and passed through a 0.45 μm Syringe Filter (Fisher Scientific). The depletion method, based on BCA™ protein assay kit (Pierce, Rockford, Ill.) was used to calculate the amount of CBD-CA adsorbed to the cellulose. Each measurement was done in triplicate.

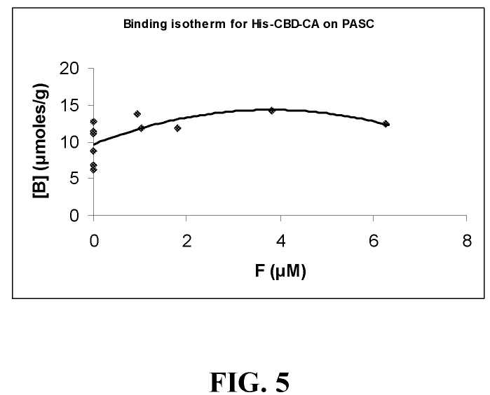

Results: Binding isotherm for His-CBD-CA was determined on PASC. Because the presence of a His tag does not affect the binding isotherm of CBD (Lehtio, J., J. Sugiyama, M. Gustavsson, L. Fransson, M. Linder, and T. T. Teeri. The binding specificity and affinity determinants of family 1 and family 3 cellulose binding modules. Proc Natl Acad Sci USA. 100:484-489, 2003), His tag-CBD-CA was used directly without excision of His-Tag for binding assay. FIG. 5 shows the binding isotherm for His-CBD-CA on PASC in 50 mM phosphate buffer at pH 8.0 and 22° C. Initial results indicate that binding is very strong.

### Alternate Embodiments

Other strategies including choice of promoter for gene expression and choice of E coli host strain as well as the co-expression of molecular chaperones and use of fusion partners for enhancing protein solubility can be explored for optimization of E. coli for production of proteins in soluble form. It may also be possible to use B. subtilis, or other hosts as a host cell for expression of CA-CBD.

B. subtilis and other bacilli have been used extensively for large scale applications of many commercial high volume and low value enzymes. The genetics of B. subtilis is also well understood and indeed it is the best-studied Gram-positive bacterium. A key advantage of B. subtilis verus E. coli for protein expression is its well-characterized protein secretion pathways that enable accumulation of some proteins at very high levels (several grams/liter of culture) in the culture fluid. One possible disadvantage of B. subtilis is the extracellular proteases which could degrade the secreted target protein. To minimize the extracellular protein degradation, one can employ the use of strains deficient in certain proteases, such as B. subtilis strain WB600. This strain is deficient in six major extracellular proteases including protease A, subtilisin, extracellular protease, metalloprotease, bacillopeptidase F, and neutral protease B. Strain WB600 displays only about 0.3% of the wild-type extracellular protease activity and thus effectively prevents degradation of extracellular proteins. For example, by using the stain WB600, substantial enhancement in beta-lactamase production has been reported.

Various vectors may be employed with B subtilis. One suitable plasmid pREP9 provides the requirements for cloning and expressing CA gene in B. subtilis. This plasmid carries replication origins for B. subtilis and E coli, chloramphenicol and kanamycin resistance genes, the gene for the E coli lac repressor gene, and a chimeric promoter, P_{N25/O}, consisting of the lac operator region fused to the P_{N25} promoter from bacteriophage T5. Genes cloned behind this promoter have been shown to be inducible by IPTG (isopropyl-b-D-thiogalactopyronoside). We have used a similar system comprised of plasmid p602/19 (was kindely provided to us by Dr. LeGrice) which contains the cat gene downstream of a strong T5 promoter controlled by lac operon regulatory system.

The CA gene may be cloned using PCR primers to amplify the gene and provide restriction sites to allow insertion of a consensus secretory signal peptide followed by the CA gene behind the P_{n25/O} promoter of pREP9. Because B. subtilis cells can be easily grown to very high cell densities in fed-batch fermentors, the CA-CBD might be able to be produced at a very low cost if is secreted at high levels.

Having described the basic concept of the invention, it will be apparent to those skilled in the art that the foregoing detailed disclosure is intended to be presented by way of example only, and is not limiting. Various alterations, improvements, and modifications are intended to be suggested and are within the scope and spirit of the present invention. Additionally, the recited order of the elements or sequences, or the use of numbers, letters or other designations therefor, is not intended to limit the claimed processes to any order except as may be specified in the claims. All ranges disclosed herein also encompass any and all possible sub-ranges and combinations of sub-ranges thereof. Any listed range can be easily recognized as sufficiently describing and enabling the same range being broken down into at least equal halves, thirds, quarters, fifths, tenths, etc. As a non-limiting example, each range discussed herein can be readily broken down into a lower third, middle third and upper third, etc. As will also be understood by one skilled in the art all language such as “up to,” “at least,” “greater than,” “less than,” and the like refer to ranges which can be subsequently broken down into sub-ranges as discussed above. Accordingly, the invention is limited only by the following claims and equivalents thereto.

All publications and patent documents cited in this application are incorporated by reference in their entirety for all purposes to the same extent as if each individual publication or patent document were so individually denoted.

We claim the following:

1. A fusion polypeptide encoding at least one carbonic anhydrase and at least one heterologous amino acid sequence wherein the heterologous amino acid sequence is a polypeptide that binds to cellulose.

2. The fusion polypeptide of claim 1, wherein the heterologous amino acid sequence is a cellulose binding domain.

3. The fusion polypeptide of claim 1, wherein the heterologous amino acid sequence has an amino acid sequence of SEQ ID NO: 22 or a functional equivalent thereof.

4. The fusion polypeptide of claim 1, wherein the carbonic anhydrase is a mature form of carbonic anhydrase.

5. The fusion polypeptide of claim 1, wherein the carbonic anhydrase has a polypeptide sequence of SEQ ID NO: 11 or a functional equivalent thereof.

6. The fusion polypeptide of claim 1, wherein the carbonic anhydrase is a mature form of carbonic anhydrase and the heterologous amino acid sequence is a cellulose binding domain.

7. The fusion polypeptide of claim 1, wherein the carbonic anhydrase has a polypeptide sequence of SEQ ID NO: 11 or a functional equivalent thereof and the heterologous amino acid sequence has an amino acid sequence of SEQ ID NO: 22 or a functional equivalent thereof.

8. The fusion polypeptide of claim 1, wherein the fusion polypeptide has an amino acid sequence comprising the SEQ ID NO.8 or a functional equivalent thereof.

9. The fusion polypeptide of claim 1, wherein the fusion polypeptide has an amino acid sequence at least 95% identical to SEQ ID NO.8.

10. The fusion polypeptide of claim 1, wherein the fusion polypeptide has an amino acid sequence comprising SEQ ID NO.8.

11. The fusion polypeptide of claim 1, wherein the polypeptide is a biologically active fragment having an amino acid sequence comprising at least 350 continuous amino acids of SEQ ID NO: 8.

12. An isolated nucleic acid comprising a polynucleotide encoding the fusion polypeptide of claim 1.

13. The nucleic acid of claim 12, wherein the fusion polypeptide has an amino acid sequence at least 95% identical to SEQ ID NO.8.

14. The nucleic acid of claim 12, wherein the fusion polypeptide has an amino acid sequence comprising SEQ ID NO.8 or a functional equivalent thereof.

15. The nucleic acid of claim 12, wherein the nucleotide sequence comprises the nucleotide sequence of SEQ ID NO: 1, or a degenerate variant thereof.

16. The nucleic acid of claim 12, wherein the nucleotide sequence comprises a sequence that is at least 90% identical to SEQ ID NO: 1.

17. The nucleic acid of claim 12, wherein the nucleotide sequence comprises a sequence that is at least 95% identical to SEQ ID NO: 1.

18. The nucleic acid of claim 12, wherein the nucleotide sequence comprises at least 1000 continuous nucleotides of SEQ ID NO: 1.

19. An expression vector comprising the nucleic acid of claim 13 operably inked to an expression control sequence.

20. The expression vector of claim 19, wherein the fusion polypeptide of claim 6 is operably inked to an expression control sequence of a suitable host cell.

21. The expression vector of claim 19, wherein the fusion polypeptide of claim 6 is operably inked to an expression control sequence of a suitable host cell, wherein the suitable host cell is E. coli.

22. The expression vector of claim 19, wherein the expression vector has a nucleic acid sequence of SEQ ID NO: 2 or functional equivalents thereof

23. A method for removing CO₂ from a gas stream comprising:
a. providing a fusion polypeptide with carbonic anhydrase activity and the ability to bind to cellulose;
b. immobilizing the fusion polypeptide onto a cellulose containing support forming an activated support,
c. contacting the activated support with a CO₂ containing gas or gas stream, wherein the fusion polypeptide reacts with at least a portion of the CO₂ in the gas or gas stream forming one or more reaction products.

24. The method of claim 23, wherein one or more of the reaction products are selected from a group consisting of: carbonate ions, bicarbonate ions, carbonate, bicarbonate and combinations thereof.

25. The method of claim 23, further comprising stabilizing the reaction products.

26. The method of claim 23, further comprising exposing the one or more reaction products to an alkali metal, alkali earth metal, alkali earth metal ion, mineral, mineral ion, compounds containing such, and combinations thereof.

27. The method of claim 23, further comprising the step of exposing the reaction product with magnesium or calcium containing substances or combinations thereof.

28. The method of claim 23, wherein the fusion polypeptide is that of claim 2.


SEQUENCE LISTING (25 sequences)

SEQ ID NO: 1 - DNA, 1248 bp, Artificial
  Artifically Created Sequence Encoding Hybrid Carbonic Anhydrase-Celluose Binding Enzyme
gaattctacaacagcaatccttcagatactactaactcaatcaatcctcagttcaaggttactaataccggaagcagtgcaattgatttgtccaaactcacattgagatattattatacagtagacggacagaaagatcagaccttctggtgtgaccatgctgcaataatcggcagtaacggcagctacaacggaattacttcaaatgtaaaaggaacatttgtaaaaatgagttcctcaacaaataacgcagacacctaccttgaaataagctttacaggcggaactcttgaaccgggtgcacatgttcagatacaaggtagatttgcaaagaatgactggagtaactatacacagtcaaatgactactcattcaagtctgcttcacagtttgttgaatgggatcaggtaacagcatacttgaacggtgttcttgtatggggtaaagaacccggtggcagtgtagtaccatcaacacagcctgtaacaacaccacctgcaacaacaaaaccacctgcaacaacaaaaccacctgcaacaacaataccgccgtcagatgatagatctcacggcaatcacacccattggggctataccggacacgactctcccgaaagctggggcaatctgtcagaagaattccgtttgtgctccaccggcaaaaaccaatctccggtaaacattaccgaaaccgtttccggcaaactgcccgccatcaaagtcaattacaaaccgagtatggttgacgtggaaaacaacggccacaccattcaggtcaattatcccgaaggcggcaataccctgaccgtgaacggccgcacctataccctgaaacagttccacttccacgtgccgagcgaaaaccaaatcaaaggccgcactttcccgatggaagctcacttcgtccacttagacgaaaacaaacagcctttagtattagccgtgctgtatgaagccggcaaaaccaacggccgcctgtcttccatctggaacgtcatgccgatgaccgcaggaaaagtgaaactcaaccaaccgttcgacgcatccaccctactgccgaaacggttgaaatactaccgctttgccggttcgctgaccacgccgccgtgcacagagggcgtatcatggttggtgttgaaaacttatgaccacatcgaccaagcgcaagcggaaaaattcacccgcgccgtcggttcggaaaacaaccgccccgtacagcctctgaatgcacgtgtagttattgaataa

SEQ ID NO: 2 - DNA, 1347 bp, Artificial
  Artifically Created Sequence Encoding Hybrid Carbonic Anhydrase-Celluose Binding Enzyme plasmid DNA
atgcggggttctcatcatcatcatcatcatggtatggctagcatgactggtggacagcaaatgggtcgggatctgtacgacgatgacgataaggatccggaattctacaacagcaatccttcagatactactaactcaatcaatcctcagttcaaggttactaataccggaagcagtgcaattgatttgtccaaactcacattgagatattattatacagtagacggacagaaagatcagaccttctggtgtgaccatgctgcaataatcggcagtaacggcagctacaacggaattacttcaaatgtaaaaggaacatttgtaaaaatgagttcctcaacaaataacgcagacacctaccttgaaataagctttacaggcggaactcttgaaccgggtgcacatgttcagatacaaggtagatttgcaaagaatgactggagtaactatacacagtcaaatgactactcattcaagtctgcttcacagtttgttgaatgggatcaggtaacagcatacttgaacggtgttcttgtatggggtaaagaacccggtggcagtgtagtaccatcaacacagcctgtaacaacaccacctgcaacaacaaaaccacctgcaacaacaaaaccacctgcaacaacaataccgccgtcagatgatagatctcacggcaatcacacccattggggctataccggacacgactctcccgaaagctggggcaatctgtcagaagaattccgtttgtgctccaccggcaaaaaccaatctccggtaaacattaccgaaaccgtttccggcaaactgcccgccatcaaagtcaattacaaaccgagtatggttgacgtggaaaacaacggccacaccattcaggtcaattatcccgaaggcggcaataccctgaccgtgaacggccgcacctataccctgaaacagttccacttccacgtgccgagcgaaaaccaaatcaaaggccgcactttcccgatggaagctcacttcgtccacttagacgaaaacaaacagcctttagtattagccgtgctgtatgaagccggcaaaaccaacggccgcctgtcttccatctggaacgtcatgccgatgaccgcaggaaaagtgaaactcaaccaaccgttcgacgcatccaccctactgccgaaacggttgaaatactaccgctttgccggttcgctgaccacgccgccgtgcacagagggcgtatcatggttggtgttgaaaacttatgaccacatcgaccaagcgcaagcggaaaaattcacccgcgccgtcggttcggaaaacaaccgccccgtacagcctctgaatgcacgtgtagttattgaataa

SEQ ID NO: 3 - DNA, 30 bp, Artificial
  Cellulose Binding Domain F-primer
gcgggatccggaattctacaacagcaatcc

SEQ ID NO: 4 - DNA, 28 bp, Artificial
  Cellulose Binding Domain r-primer
atttgcagatctatcatctgacggcggt

SEQ ID NO: 5 - DNA, 29 bp, Artificial
  Carboninc Anhydrase f-primer
atttgcagatctcacggcaatcacaccca

SEQ ID NO: 6 - DNA, 29 bp, Artificial
  Carbonic Anhydrase r-primer
acggccatggttattcaataactacacgt

SEQ ID NO: 7 - RNA, 1248 nt, Artificial
  Artificial Carbonic Anhydrase-Cellulose Binding Domain RNA
gaauucuacaacagcaauccuucagauacuacuaacucaaucaauccucaguucaagguuacuaauaccggaagcagugcaauugauuuguccaaacucacauugagauauuauuauacaguagacggacagaaagaucagaccuucuggugugaccaugcugcaauaaucggcaguaacggcagcuacaacggaauuacuucaaauguaaaaggaacauuuguaaaaaugaguuccucaacaaauaacgcagacaccuaccuugaaauaagcuuuacaggcggaacucuugaaccgggugcacauguucagauacaagguagauuugcaaagaaugacuggaguaacuauacacagucaaaugacuacucauucaagucugcuucacaguuuguugaaugggaucagguaacagcauacuugaacgguguucuuguaugggguaaagaacccgguggcaguguaguaccaucaacacagccuguaacaacaccaccugcaacaacaaaaccaccugcaacaacaaaaccaccugcaacaacaauaccgccgucagaugauagaucucacggcaaucacacccauuggggcuauaccggacacgacucucccgaaagcuggggcaaucugucagaagaauuccguuugugcuccaccggcaaaaaccaaucuccgguaaacauuaccgaaaccguuuccggcaaacugcccgccaucaaagucaauuacaaaccgaguaugguugacguggaaaacaacggccacaccauucaggucaauuaucccgaaggcggcaauacccugaccgugaacggccgcaccuauacccugaaacaguuccacuuccacgugccgagcgaaaaccaaaucaaaggccgcacuuucccgauggaagcucacuucguccacuuagacgaaaacaaacagccuuuaguauuagccgugcuguaugaagccggcaaaaccaacggccgccugucuuccaucuggaacgucaugccgaugaccgcaggaaaagugaaacucaaccaaccguucgacgcauccacccuacugccgaaacgguugaaauacuaccgcuuugccgguucgcugaccacgccgccgugcacagagggcguaucaugguugguguugaaaacuuaugaccacaucgaccaagcgcaagcggaaaaauucacccgcgccgucgguucggaaaacaaccgccccguacagccucugaaugcacguguaguuauugaauaa

SEQ ID NO: 8 - protein, 415 aa, Artificial
  Artifical Carbonic Anhydrase-Cellulose Binding Domain protein
EFYNSNPSDTTNSINPQFKVTNTGSSAIDLSKLTLRYYYTVDGQKDQTFWCDHAAIIGSNGSYNGITSNVKGTFVKMSSSTNNADTYLEISFTGGTLEPGAHVQIQGRFAKNDWSNYTQSNDYSFKSASQFVEWDQVTAYLNGVLVWGKEPGGSVVPSTQPVTTPPATTKPPATTKPPATTIPPSDDRSHGNHTHWGYTGHDSPESWGNLSEEFRLCSTGKNQSPVNITETVSGKLPAIKVNYKPSMVDVENNGHTIQVNYPEGGNTLTVNGRTYTLKQFHFHVPSENQIKGRTFPMEAHFVHLDENKQPLVLAVLYEAGKTNGRLSSIWNVMPMTAGKVKLNQPFDASTLLPKRLKYYRFAGSLTTPPCTEGVSWLVLKTYDHIDQAQAEKFTRAVGSENNRPVQPLNARVVIE

SEQ ID NO: 9 - protein, 448 aa, Artificial
  pRSET-CBD-CA Protein Sequence
MRGSHHHHHHGMASMTGGQQMGRDLYDDDDKDPEFYNSNPSDTTNSINPQFKVTNTGSSAIDLSKLTLRYYYTVDGQKDQTFWCDHAAIIGSNGSYNGITSNVKGTFVKMSSSTNNADTYLEISFTGGTLEPGAHVQIQGRFAKNDWSNYTQSNDYSFKSASQFVEWDQVTAYLNGVLVWGKEPGGSVVPSTQPVTTPPATTKPPATTKPPATTIPPSDDRSHGNHTHWGYTGHDSPESWGNLSEEFRLCSTGKNQSPVNITETVSGKLPAIKVNYKPSMVDVENNGHTIQVNYPEGGNTLTVNGRTYTLKQFHFHVPSENQIKGRTFPMEAHFVHLDENKQPLVLAVLYEAGKTNGRLSSIWNVMPMTAGKVKLNQPFDASTLLPKRLKYYRFAGSLTTPPCTEGVSWLVLKTYDHIDQAQAEKFTRAVGSENNRPVQPLNARVVIE

SEQ ID NO: 10 - DNA, 681 bp, Artificial
  Mature Carbonic Anhydrase-DNA
cacggcaatcacacccattggggctataccggacacgactctcccgaaagctggggcaatctgtcagaagaattccgtttgtgctccaccggcaaaaaccaatctccggtaaacattaccgaaaccgtttccggcaaactgcccgccatcaaagtcaattacaaaccgagtatggttgacgtggaaaacaacggccacaccattcaggtcaattatcccgaaggcggcaataccctgaccgtgaacggccgcacctataccctgaaacagttccacttccacgtgccgagcgaaaaccaaatcaaaggccgcactttcccgatggaagctcacttcgtccacttagacgaaaacaaacagcctttagtattagccgtgctgtatgaagccggcaaaaccaacggccgcctgtcttccatctggaacgtcatgccgatgaccgcaggaaaagtgaaactcaaccaaccgttcgacgcatccaccctactgccgaaacggttgaaatactaccgctttgccggttcgctgaccacgccgccgtgcacagagggcgtatcatggttggtgttgaaaacttatgaccacatcgaccaagcgcaagcggaaaaattcacccgcgccgtcggttcggaaaacaaccgccccgtacagcctctgaatgcacgtgtagttattgaataa

SEQ ID NO: 11 - protein, 226 aa, Artificial
  Mature Carbonic Anhydrase-protein Sequence
HGNHTHWGYTGHDSPESWGNLSEEFRLCSTGKNQSPVNITETVSGKLPAIKVNYKPSMVDVENNGHTIQVNYPEGGNTLTVNGRTYTLKQFHFHVPSENQIKGRTFPMEAHFVHLDENKQPLVLAVLYEAGKTNGRLSSIWNVMPMTAGKVKLNQPFDASTLLPKRLKYYRFAGSLTTPPCTEGVSWLVLKTYDHIDQAQAEKFTRAVGSENNRPVQPLNARVVIE

SEQ ID NO: 12 - DNA, 1248 bp, Artificial
  Artificial Carbonic-Cellulose Binding Domain DNA alternative sequence
gaattctataacagcaatccttcagatactactaactcaatcaatcctcagttcaaggttactaataccggaagcagtgcaattgatttgtccaaactcacattgagatattattatacagtagacggacagaaagatcagaccttctggtgtgaccatgctgcaataatcggcagtaacggcagctacaacggaattacttcaaatgtaaaaggaacatttgtaaaaatgagttcctcaacaaataacgcagacacctaccttgaaataagctttacaggcggaactcttgaaccgggtgcacatgttcagatacaaggtagatttgcaaagaatgactggagtaactatacacagtcaaatgactactcattcaagtctgcttcacagtttgttgaatgggatcaggtaacagcatacttgaacggtgttcttgtatggggtaaagaacccggtggcagtgtagtaccatcaacacagcctgtaacaacaccacctgcaacaacaaaaccacctgcaacaacaaaaccacctgcaacaacaataccgccgtcagatgatagatctcacggcaatcacacccattggggctataccggacacgactctcccgaaagctggggcaatctgtcagaagaattccgtttgtgctccaccggcaaaaaccaatctccggtaaacattaccgaaaccgtttccggcaaactgcccgccatcaaagtcaattacaaaccgagtatggttgacgtggaaaacaacggccacaccattcaggtcaattatcccgaaggcggcaataccctgaccgtgaacggccgcacctataccctgaaacagttccacttccacgtgccgagcgaaaaccaaatcaaaggccgcactttcccgatggaagctcacttcgtccacttagacgaaaacaaacagcctttagtattagccgtgctgtatgaagccggcaaaaccaacggccgcctgtcttccatctggaacgtcatgccgatgaccgcaggaaaagtgaaactcaaccaaccgttcgacgcatccaccctactgccgaaacggttgaaatactaccgctttgccggttcgctgaccacgccgccgtgcacagagggcgtatcatggttggtgttgaaaacttatgaccacatcgaccaagcgcaagcggaaaaattcacccgcgccgtcggttcggaaaacaaccgccccgtacagcctctgaatgcacgtgtagttattgaataa

SEQ ID NO: 13 - DNA, 1248 bp, Artificial
  Carbonic Ahydrase-Cellulose Binding Domain alt seq
gaattctacaacagcaatccttcagatactactaactcaatcaatcctcagttcaaggttactaataccggaagcagtgcaattgatttgtccaaactcacattgagatattattatacagtagacggacagaaagatcagaccttctggtgtgaccatgctgcaataatcggcagtaacggcagctacaacggaattacttcaaatgtaaaaggaacatttgtaaaaatgagttcctcaacaaataacgcagacacctaccttgaaataagctttacaggcggaactcttgaaccgggtgcacatgttcagatacaaggtagatttgcaaagaatgactggagtaactatacacagtcaaatgactactcattcaagtctgcttcacagtttgttgaatgggatcaggtaacagcatacttgaacggtgttcttgtatggggtaaagaacccggtggcagtgtagtaccatcaacacagcctgtaacaacaccacctgcaacaacaaaaccacctgcaacaacaaaaccacctgcaacaacaataccgccgtcagatgatagatctcacggcaatcacacccattggggctataccggacacgactctcccgaaagctggggcaatctgtcagaagaattccgtttgtgctccaccggcaaaaaccaatctccggtaaacattaccgaaaccgtttccggcaaactgcccgccatcaaagtcaattacaaaccgagtatggttgacgtggaaaacaacggccacaccattcaggtcaattatcccgaaggcggcaataccctgaccgtgaacggccgcaactataccctgaaacagttccacttccacgtgccgagcgaaaaccaaatcaaaggccgcactttcccgatggaagctcacttcgtccacttagacgaaaacaaacagcctttagtattagccgtgctgtatgaagccggcaaaaccaacggccgcctgtcttccatctggaacgtcatgccgatgaccgcaggaaaagtgaaactcaaccaaccgttcgacgcatccaccctactgccgaaacggttgaaatactaccgctttgccggttcgctgaccacgccgccgtgcacagagggcgtatcatggttggtgttgaaaacttatgaccacatcgaccaagcgcaagcggaaaaattcacccgcgccgtcggttcggaaaacaaccgccccgtacagcctctgaatgcacgtgtagttattgaataa

SEQ ID NO: 14 - protein, 415 aa, Artificial
  Carbonic Anhydrase-Cellulose Binding Domain alt protein seq
EFYNSNPSDTTNSINPQFKVTNTGSSAIDLSKLTLRYYYTVDGQKDQTFWCDHAAIIGSNGSYNGITSNVKGTFVKMSSSTNNADTYLEISFTGGTLEPGAHVQIQGRFAKNDWSNYTQSNDYSFKSASQFVEWDQVTAYLNGVLVWGKEPGGSVVPSTQPVTTPPATTKPPATTKPPATTIPPSDDRSHGNHTHWGYTGHDSPESWGNLSEEFRLCSTGKNQSPVNITETVSGKLPAIKVNYKPSMVDVENNGHTIQVNYPEGGNTLTVNGRNYTLKQFHFHVPSENQIKGRTFPMEAHFVHLDENKQPLVLAVLYEAGKTNGRLSSIWNVMPMTAGKVKLNQPFDASTLLPKRLKYYRFAGSLTTPPCTEGVSWLVLKTYDHIDQAQAEKFTRAVGSENNRPVQPLNARVVIE

SEQ ID NO: 15 - DNA, 681 bp, Artificial; identical to SEQ ID NO: 10
  Carbonic Anhydrase alternative seq

SEQ ID NO: 16 - DNA, 681 bp, Artificial
  Carbonic Anhydrase alt seq
cacggcaatcacacccattggggctataccggacacgactctcccgaaagctggggcaatctgtcagaagaattccgtttgtgctccaccggcaaaaaccaatctccggtaaacattaccgaaaccgtttccggcaaactgcccgccatcaaagtcaattacaaaccgagtatggttgacgtggaaaacaacggccacaccattcaggtcaattatcccgaaggcggcaataccctgaccgtgaacggccgcacctataccgtgaaacagttccacttccacgtgccgagcgaaaaccaaatcaaaggccgcactttcccgatggaagctcacttcgtccacttagacgaaaacaaacagcctttagtattagccgtgctgtatgaagccggcaaaaccaacggccgcctgtcttccatctggaacgtcatgccgatgaccgcaggaaaagtgaaactcaaccaaccgttcgacgcatccaccctactgccgaaacggttgaaatactaccgctttgccggttcgctgaccacgccgccgtgcacagagggcgtatcatggttggtgttgaaaacttatgaccacatcgaccaagcgcaagcggaaaaattcacccgcgccgtcggttcggaaaacaaccgccccgtacagcctctgaatgcacgtgtagttattgaataa

SEQ ID NO: 17 - protein, 226 aa, Artificial
  Carbonic Anhydrase alt protein
HGNHTHWGYTGHDSPESWGNLSEEFRLCSTGKNQSPVNITETVSGKLPAIKVNYKPSMVDVENNGHTIQVNYPEGGNTLTVNGRTYTVKQFHFHVPSENQIKGRTFPMEAHFVHLDENKQPLVLAVLYEAGKTNGRLSSIWNVMPMTAGKVKLNQPFDASTLLPKRLKYYRFAGSLTTPPCTEGVSWLVLKTYDHIDQAQAEKFTRAVGSENNRPVQPLNARVVIE

SEQ ID NO: 18 - DNA, 1347 bp, Artificial
  Carbonic Anhydrase-Cellulose Binding Domain-plasmid alt
atgcggggttcccatcatcatcatcatcatggtatggctagcatgactggtggacagcaaatgggtcgggatctgtacgacgatgacgataaggatccggaattctacaacagcaatccttcagatactactaactcaatcaatcctcagttcaaggttactaataccggaagcagtgcaattgatttgtccaaactcacattgagatattattatacagtagacggacagaaagatcagaccttctggtgtgaccatgctgcaataatcggcagtaacggcagctacaacggaattacttcaaatgtaaaaggaacatttgtaaaaatgagttcctcaacaaataacgcagacacctaccttgaaataagctttacaggcggaactcttgaaccgggtgcacatgttcagatacaaggtagatttgcaaagaatgactggagtaactatacacagtcaaatgactactcattcaagtctgcttcacagtttgttgaatgggatcaggtaacagcatacttgaacggtgttcttgtatggggtaaagaacccggtggcagtgtagtaccatcaacacagcctgtaacaacaccacctgcaacaacaaaaccacctgcaacaacaaaaccacctgcaacaacaataccgccgtcagatgatagatctcacggcaatcacacccattggggctataccggacacgactctcccgaaagctggggcaatctgtcagaagaattccgtttgtgctccaccggcaaaaaccaatctccggtaaacattaccgaaaccgtttccggcaaactgcccgccatcaaagtcaattacaaaccgagtatggttgacgtggaaaacaacggccacaccattcaggtcaattatcccgaaggcggcaataccctgaccgtgaacggccgcacctataccctgaaacagttccacttccacgtgccgagcgaaaaccaaatcaaaggccgcactttcccgatggaagctcacttcgtccacttagacgaaaacaaacagcctttagtattagccgtgctgtatgaagccggcaaaaccaacggccgcctgtcttccatctggaacgtcatgccgatgaccgcaggaaaagtgaaactcaaccaaccgttcgacgcatccaccctactgccgaaacggttgaaatactaccgctttgccggttcgctgaccacgccgccgtgcacagagggcgtatcatggttggtgttgaaaacttatgaccacatcgaccaagcgcaagcggaaaaattcacccgcgccgtcggttcggaaaacaaccgccccgtacagcctctgaatgcacgtgtagttattgaataa

SEQ ID NO: 19 - DNA, 1347 bp, Artificial
  Carbonic Anhydrase-Cellulose Binding Domain-plasmid alt
atgcggggttgtcatcatcatcatcatcatggtatggctagcatgactggtggacagcaaatgggtcgggatctgtacgacgatgacgataaggatccggaattctacaacagcaatccttcagatactactaactcaatcaatcctcagttcaaggttactaataccggaagcagtgcaattgatttgtccaaactcacattgagatattattatacagtagacggacagaaagatcagaccttctggtgtgaccatgctgcaataatcggcagtaacggcagctacaacggaattacttcaaatgtaaaaggaacatttgtaaaaatgagttcctcaacaaataacgcagacacctaccttgaaataagctttacaggcggaactcttgaaccgggtgcacatgttcagatacaaggtagatttgcaaagaatgactggagtaactatacacagtcaaatgactactcattcaagtctgcttcacagtttgttgaatgggatcaggtaacagcatacttgaacggtgttcttgtatggggtaaagaacccggtggcagtgtagtaccatcaacacagcctgtaacaacaccacctgcaacaacaaaaccacctgcaacaacaaaaccacctgcaacaacaataccgccgtcagatgatagatctcacggcaatcacacccattggggctataccggacacgactctcccgaaagctggggcaatctgtcagaagaattccgtttgtgctccaccggcaaaaaccaatctccggtaaacattaccgaaaccgtttccggcaaactgcccgccatcaaagtcaattacaaaccgagtatggttgacgtggaaaacaacggccacaccattcaggtcaattatcccgaaggcggcaataccctgaccgtgaacggccgcacctataccctgaaacagttccacttccacgtgccgagcgaaaaccaaatcaaaggccgcactttcccgatggaagctcacttcgtccacttagacgaaaacaaacagcctttagtattagccgtgctgtatgaagccggcaaaaccaacggccgcctgtcttccatctggaacgtcatgccgatgaccgcaggaaaagtgaaactcaaccaaccgttcgacgcatccaccctactgccgaaacggttgaaatactaccgctttgccggttcgctgaccacgccgccgtgcacagagggcgtatcatggttggtgttgaaaacttatgaccacatcgaccaagcgcaagcggaaaaattcacccgcgccgtcggttcggaaaacaaccgccccgtacagcctctgaatgcacgtgtagttattgaataa

SEQ ID NO: 20 - protein, 448 aa, Artificial
  Carbonic ANhydrase-Cellulose Binding Domain-plasmid alt prt
MRGCHHHHHHGMASMTGGQQMGRDLYDDDDKDPEFYNSNPSDTTNSINPQFKVTNTGSSAIDLSKLTLRYYYTVDGQKDQTFWCDHAAIIGSNGSYNGITSNVKGTFVKMSSSTNNADTYLEISFTGGTLEPGAHVQIQGRFAKNDWSNYTQSNDYSFKSASQFVEWDQVTAYLNGVLVWGKEPGGSVVPSTQPVTTPPATTKPPATTKPPATTIPPSDDRSHGNHTHWGYTGHDSPESWGNLSEEFRLCSTGKNQSPVNITETVSGKLPAIKVNYKPSMVDVENNGHTIQVNYPEGGNTLTVNGRTYTLKQFHFHVPSENQIKGRTFPMEAHFVHLDENKQPLVLAVLYEAGKTNGRLSSIWNVMPMTAGKVKLNQPFDASTLLPKRLKYYRFAGSLTTPPCTEGVSWLVLKTYDHIDQAQAEKFTRAVGSENNRPVQPLNARVVIE

SEQ ID NO: 21 - DNA, 563 bp, Artificial
  Cellulose Binding Domain
gaattctacaacagcaatccttcagatactactaactcaatcaatcctcagttcaaggttactaataccggaagcagtgcaattgatttgtccaaactcacattgagatattattatacagtagacggacagaaagatcagaccttctggtgtgaccatgctgcaataatcggcagtaacggcagctacaacggaattacttcaaatgtaaaaggaacatttgtaaaaatgagttcctcaacaaataacgcagacacctaccttgaaataagctttacaggcggaactcttgaaccgggtgcacatgttcagatacaaggtagatttgcaaagaatgactggagtaactatacacagtcaaatgactactcattcaagtctgcttcacagtttgttgaatgggatcaggtaacagcatacttgaacggtgttcttgtatggggtaaagaacccggtggcagtgtagtaccatcaacacagcctgtaacaacaccacctgcaacaacaaaaccacctgcaacaacaaaaccacctgcaacaacaataccgccgtcagatgatcc

SEQ ID NO: 22 - protein, 187 aa, Artificial
  Cellulose Binding Domain prt
EFYNSNPSDTTNSINPQFKVTNTGSSAIDLSKLTLRYYYTVDGQKDQTFWCDHAAIIGSNGSYNGITSNVKGTFVKMSSSTNNADTYLEISFTGGTLEPGAHVQIQGRFAKNDWSNYTQSNDYSFKSASQFVEWDQVTAYLNGVLVWGKEPGGSVVPSTQPVTTPPATTKPPATTKPPATTIPPSDD

SEQ ID NO: 23 - DNA, 563 bp, Artificial
  Cellulose Binding Domain alt seq
gagttctacaacagcaatccttcagatactactaactcaatcaatcctcagttcaaggttactaataccggaagcagtgcaattgatttgtccaaactcacattgagatattattatacagtagacggacagaaagatcagaccttctggtgtgaccatgctgcaataatcggcagtaacggcagctacaacggaattacttcaaatgtaaaaggaacatttgtaaaaatgagttcctcaacaaataacgcagacacctaccttgaaataagctttacaggcggaactcttgaaccgggtgcacatgttcagatacaaggtagatttgcaaagaatgactggagtaactatacacagtcaaatgactactcattcaagtctgcttcacagtttgttgaatgggatcaggtaacagcatacttgaacggtgttcttgtatggggtaaagaacccggtggcagtgtagtaccatcaacacagcctgtaacaacaccacctgcaacaacaaaaccacctgcaacaacaaaaccacctgcaacaacaataccgccgtcagatgatcc

SEQ ID NO: 24 - DNA, 563 bp, Artificial
  Cellulose Binding Domain alt seq
gaattctacaccagcaatccttcagatactactaactcaatcaatcctcagttcaaggttactaataccggaagcagtgcaattgatttgtccaaactcacattgagatattattatacagtagacggacagaaagatcagaccttctggtgtgaccatgctgcaataatcggcagtaacggcagctacaacggaattacttcaaatgtaaaaggaacatttgtaaaaatgagttcctcaacaaataacgcagacacctaccttgaaataagctttacaggcggaactcttgaaccgggtgcacatgttcagatacaaggtagatttgcaaagaatgactggagtaactatacacagtcaaatgactactcattcaagtctgcttcacagtttgttgaatgggatcaggtaacagcatacttgaacggtgttcttgtatggggtaaagaacccggtggcagtgtagtaccatcaacacagcctgtaacaacaccacctgcaacaacaaaaccacctgcaacaacaaaaccacctgcaacaacaataccgccgtcagatgatcc

SEQ ID NO: 25 - protein, 187 aa, Artificial
  Celluose Binding Domain alt prt seq
EFYTSNPSDTTNSINPQFKVTNTGSSAIDLSKLTLRYYYTVDGQKDQTFWCDHAAIIGSNGSYNGITSNVKGTFVKMSSSTNNADTYLEISFTGGTLEPGAHVQIQGRFAKNDWSNYTQSNDYSFKSASQFVEWDQVTAYLNGVLVWGKEPGGSVVPSTQPVTTPPATTKPPATTKPPATTIPPSDD